(19) United States
(12) Patent Application Publication
(10) Pub. No.: US 2013/0098359 A1
(43) Pub. Date: Apr. 25, 2013

(54) NARES MASK AND SUPPORT APPARATUS

(71) Applicant: Somnetics Global Pte, Ltd., Singapore (SG)

(72) Inventors: Eric Allan Becker, Brooklyn Park, MN (US); Bruce R. Bowman, Eden Prairie, MN (US)

(73) Assignee: SOMNETICS GLOBAL PTE. LTD., Singapore (SG)

(21) Appl. No.: 13/651,820

(22) Filed: Oct. 15, 2012

Related U.S. Application Data
(60) Provisional application No. 61/549,989, filed on Oct. 21, 2011.

(51) Int. Cl. A61M 16/06; A61M 16/16

(52) U.S. Cl. 128/201.13; 128/205.25

(57) ABSTRACT

Embodiments of the present invention provide a nares mask and support apparatus (e.g., for use with a positive airway pressure therapy system) that may generate the desired mask retaining force while reducing or even eliminating the need for supplemental facial contact elements such as face-contacting straps or bands. The apparatus may include a first biasing element that biases a nares interface in a direction generally orthogonal to and towards the user's face. A second biasing element may bias the nares interface in a direction generally parallel to the face and towards the user's forehead. The biasing forces applied by the first and second biasing elements may thus produce a resultant force that biases the nares interface in a direction generally aligned with an axis of the nares.

### RELATED APPLICATIONS

This application claims the benefit of U.S. Provisional Application No. 61/549,989, filed Oct. 21, 2011, the content of which is incorporated herein by reference in its entirety.

### TECHNICAL FIELD

Embodiments of the present invention relate generally to respiratory apparatus, systems, and methods and, more particularly, to a nares mask and support apparatus for use during the administration of positive airway pressure therapy to a user.

### BACKGROUND

Positive airway pressure therapy may be used to treat a variety of respiratory issues including, but not limited to, obstructive sleep apnea, asthma, bronchitis, chronic obstructive pulmonary disease (COPD), snoring, congestive heart failure, and other ailments. These therapies typically pressurize an airway of the user to a pressure in the range of about 4-30 centimeters (cm) of water (e.g., about 4-20 cm of water) or more by way of the nose and/or mouth. Providing positive air pressure to a user's airways has been found to assist in maintaining airway patency during sleep. In addition to conventional continuous positive airway pressure (CPAP) therapy, patients with respiratory insufficiency may benefit from other types of ventilation assistance such as Bi-level positive airway pressure (BiPAP) therapy, Proportional Assist Ventilation (PAV) therapy, and/or other devices/methods that alter delivered pressure based on patient breathing patterns.

Many systems capable of providing positive airway pressure therapy are currently available, e.g., for the treatment of chronic sleep apnea, COPD, and snoring. These systems generally include a source of pressurized air, a tube leading from the source of pressurized air to the user, and a mask or user interface for connecting the system to the airway of the user.

One problem faced by manufacturers of positive airway pressure therapy devices is that an adequate seal is needed between the user and the user interface to minimize excessive air leaks. For example, one type of interface is a nares mask. The nares mask may include protruding portions that abut or extend into each naris. Each protruding portion may include a soft flexible pillow or prong that may seal in or around its respective naris once the system is pressurized. Nares masks may provide various advantages including, for example, smaller mask facial contact (the area of the seal) as compared to a full nasal mask or a full face mask. Such a mask may be not only more comfortable, but may also necessitate lower mask retention forces than these other mask types.

Even though retention forces may be relatively smaller with the use of a nares mask, adequate sealing relies, at least in part, on those forces being applied at a proper magnitude and direction. Some known nares mask configurations may rely on the inherent flexibility of one of more components of the nares mask to apply the desired force. However, such systems may not always yield the desired force magnitude and direction and may thus be prone to undesirable leaks. Other nares mask configurations utilize straps that pass over the face of the user and wrap around the head to provide the desired retention force. Such straps, while reliable, may be uncomfortable for some users, potentially leading to a lack of therapeutic compliance.

### SUMMARY

Accordingly, a need exists for a nares mask apparatus/method that permits application and control of forces to position and seal the mask to nares of a user, preferably while minimizing, or even eliminating, the use of additional face-contacting straps.

In one embodiment, a support apparatus for use with a nares interface configured to engage nares of a user is provided. The support apparatus includes one or more biasing elements configured to bias the nares interface in a direction generally aligned with an axis of the nares, wherein the support apparatus is configured to contact the user's face only at locations in and/or around the user's nares.

In another embodiment, a nares mask and support apparatus is provided that includes a mask having a nares interface configured to engage nares of a user, wherein the mask defines a passageway configured to deliver pressurized air into the flares. An anchor is also provided and configured to be secured to a scalp of the user. The apparatus also includes: an arm extending from the anchor to a housing of the mask; and a first biasing element operatively coupled to the arm, wherein the first biasing element is configured to bias the interface in a direction towards a face of the user. A second biasing element is also provided and operatively coupled to the housing, the second biasing element configured to bias the interface in a direction generally parallel to the face and towards a forehead of the user.

In yet another embodiment, a nares mask and support apparatus is provided that includes a mask housing operable for positioning adjacent a user's face, the housing coupled to a nares interface configured to engage nares of the user. The housing and the interface together define a passageway configured to deliver pressurized air into the nares. Also included are a base configured to be secured to a forehead of the user, and an arm having a first portion attached to the base and a second portion operatively attached to the housing. A first spring is interposed between the base and the first portion of the arm, wherein the first spring is operable to bias the arm and the housing towards the user's face. A second spring is interposed between the second portion of the arm and the housing, wherein the second spring is configured to bias the housing in a direction parallel to the face and towards the base.

In still yet another embodiment, a method for supporting a nares mask relative to a head of a user is provided, wherein the method includes placing a base of an anchor against a forehead of the user. The anchor also includes an arm pivotally attached to the base and extending away therefrom. The arm is coupled at an opposite end to a housing of the nares mask. The method also includes: inserting a nares interface into nares of the user; biasing the nares interface in a direction generally orthogonal to and towards a face of the user; and biasing the nares interface in a direction generally parallel to the user's face and towards the forehead.

The above summary is not intended to describe each embodiment or every implementation of the present invention. Rather, a more complete understanding of the invention will become apparent and appreciated by reference to the following Detailed Description of Exemplary Embodiments and claims in view of the accompanying figures of the drawing.

### BRIEF DESCRIPTION OF THE VIEWS OF THE DRAWING

The present invention will be further described with reference to the figures of the drawing, wherein:

FIG. 1A-1B illustrate exemplary positive airway pressure therapy systems incorporating a nares mask and support apparatus in accordance with one embodiment of the invention, wherein; FIG. 1A illustrates a body-mounted blower and FIG. 1B illustrates a remotely-located blower;

FIG. 2 is a cross sectional view of the mask and support apparatus of FIG. 1A or FIG. 1B;

FIG. 3 is a partial perspective view of the mask and support apparatus of FIG. 2;

FIG. 4 is an opposing partial perspective view of the apparatus of FIGS. 3; and

FIG. 5 is a perspective view of a portion of the mask and support apparatus of FIGS. 1A-4.

The figures are rendered primarily for clarity and, as a result, are not necessarily drawn to scale.

### DETAILED DESCRIPTION OF EXEMPLARY EMBODIMENTS

In the following detailed description of illustrative embodiments of the invention, reference is made to the accompanying figures of the drawing which form a part hereof, and in which are shown, by way of illustration, specific embodiments in which the invention may be practiced. It is to be understood that other embodiments may be utilized and structural changes may be made without departing from the scope of the present invention.

As used herein, relative terms such as “left,” “right,” “fore,” “forward,” “aft,” “rearward,” “top,” “bottom,” “upper,” “lower,” “horizontal,” “vertical,” and the like are from the perspective of the particular referenced figure. These terms are used herein to simplify the description, however, and not to limit the scope of the invention in any way.

Moreover, as used herein, the term “face” refers to the front part of a user's head extending from the eyebrows to the chin. “Crown” may be used herein to refer to the upper or top area of the head (assuming a standing position). The term “forehead” may be used to indicate the area of the head between the eyebrows and the crown. Similarly, the term “scalp” refers to the anatomical area bordered by the face anteriorly and the neck to the sides and posteriorly, i.e., that part of the user's head (including the forehead) that does not include the face. “Air” is used herein to denote a gas to be delivered to a user or patient and includes atmospheric gas, oxygen, other therapeutic gases, and combinations thereof, as well as fluids that provide therapeutic and/or other benefits (e.g., water (in liquid or vapor form) for humidification). “Nares mask” refers herein to a mask that contacts a patient only at or around the nares via nasal pillows or nasal prongs.

Embodiments of the present invention may provide a nares mask and support apparatus (e.g., for use with a positive airway pressure therapy system) that may generate the desired mask retaining force (abutting force between the mask and the user) while minimizing the amount of strapping used on, or in proximity to, the face, or even eliminating the need for any such supplemental face-contacting elements. Accordingly, masks in accordance with some embodiments of the present invention may contact the user's face only in and around the nares themselves.

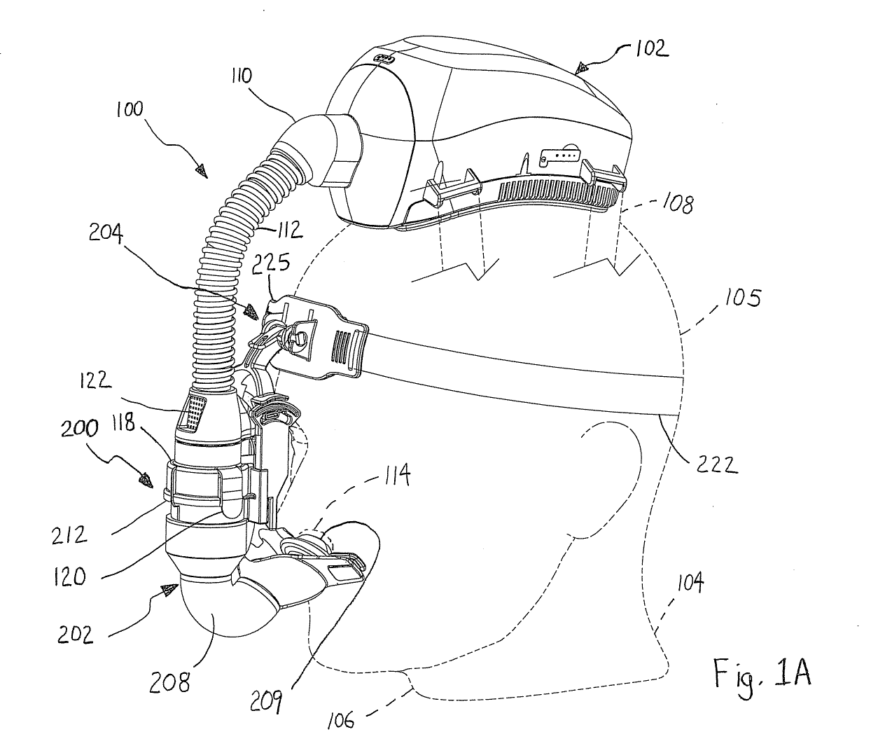

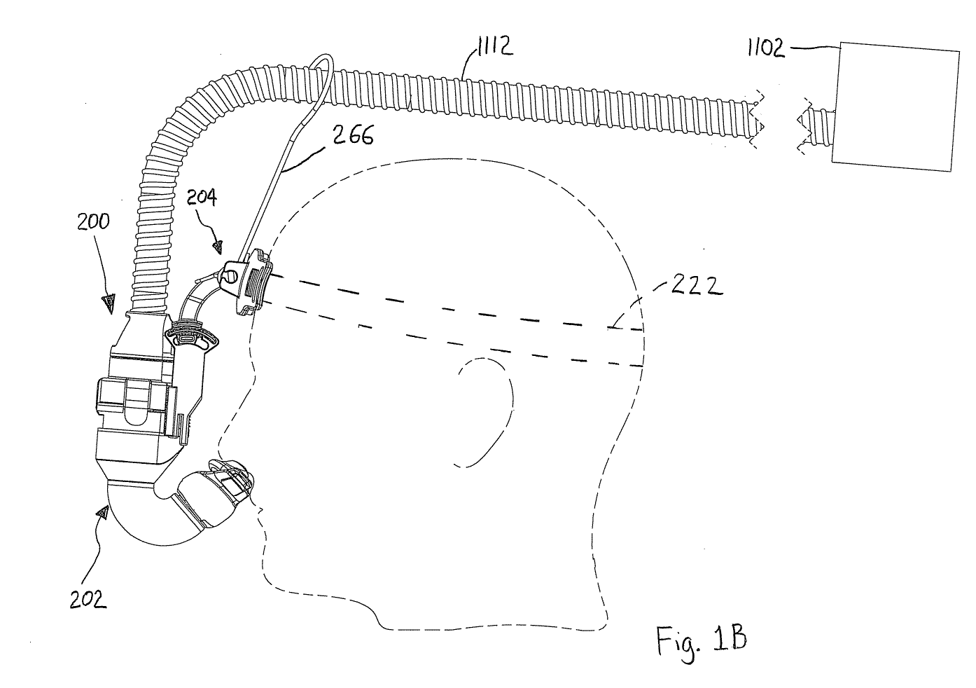

FIG. 1A illustrates a positive airway pressure system 100 in accordance with one embodiment of the invention. The system 100 may be used to deliver continuous positive airway pressure (CPAP) therapy for the treatment of obstructive sleep apnea (OSA) or other respiratory ailments. While described generally herein in the context of CPAP therapy, those of skill in the art will recognize that embodiments of the present invention may, without departing from the scope of the invention, find application to most any positive airway pressure or other respiratory therapy that requires securing a mask relative to a user's face. For example, FIG. 1B illustrates a more traditional CPAP configuration wherein the device may be located some distance from the user, e.g., often on a nightstand or the floor.

The system 100 may include a source of pressurized air, e.g., a blower 102. In one embodiment, the blower 102 may be enclosed in a housing that is securable to a user 104, e.g., to a crown of the head 105 or other portion of a user's body 106. The blower 102 may be secured to the head with one or more bands or straps 108 (only partially shown in FIG. 1A) that may wrap around the head. Such exemplary body-attached blowers are described in more detail in U.S. Pat. No. 8,020,557 and U.S. Pat. Pat. App. Pub. No. 2012-0167879. In other embodiments, the blower could be detached from the body 106 as represented by blower 1102 shown in FIG. 1B.

The blower 102 may include an inlet that draws in ambient air and then expels pressurized air through a blower outlet 110. The blower outlet 110 may be connected, via a tube or hose 112 (or hose 1112 in the case of the detached blower 1102 of FIG. 1B), to a nares mask 202. The nares mask 202 may form a portion of an exemplary nares mask and support apparatus 200 that may additionally include a mask support 204.

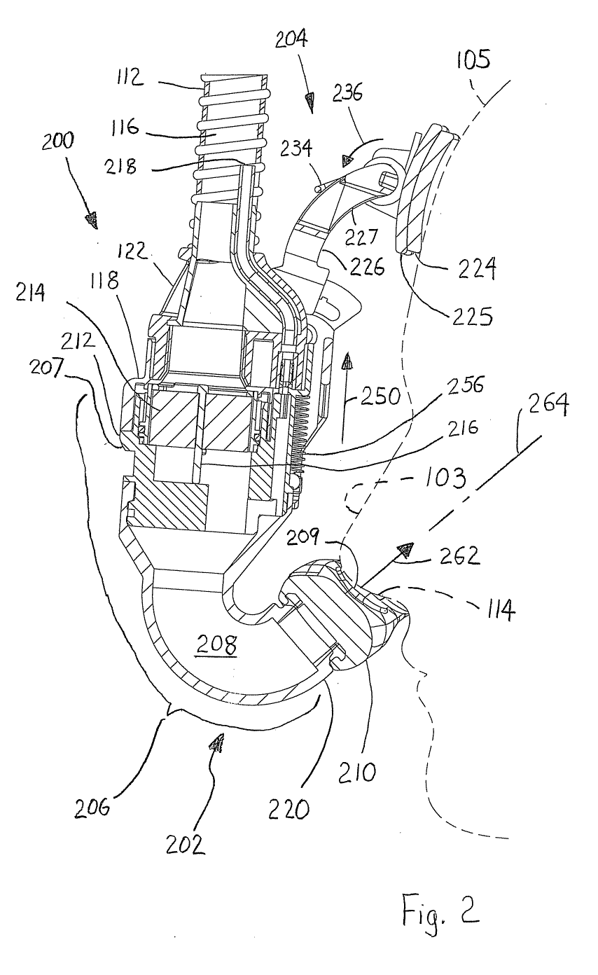

As indicated in FIG. 1A, the mask 202 may define a generally enclosed lumen or passageway 208 (see also FIG. 2) configured to deliver the pressurized air generated by the blower 102. The pressurized air may pass through the hose 112 and passageway 208 of the mask 202 and ultimately enter nares 114 of the user 104. By delivering the pressurized air in accordance with a desired profile, the system 100 may provide therapies that alleviate or eliminate OSA and other respiratory ailments.

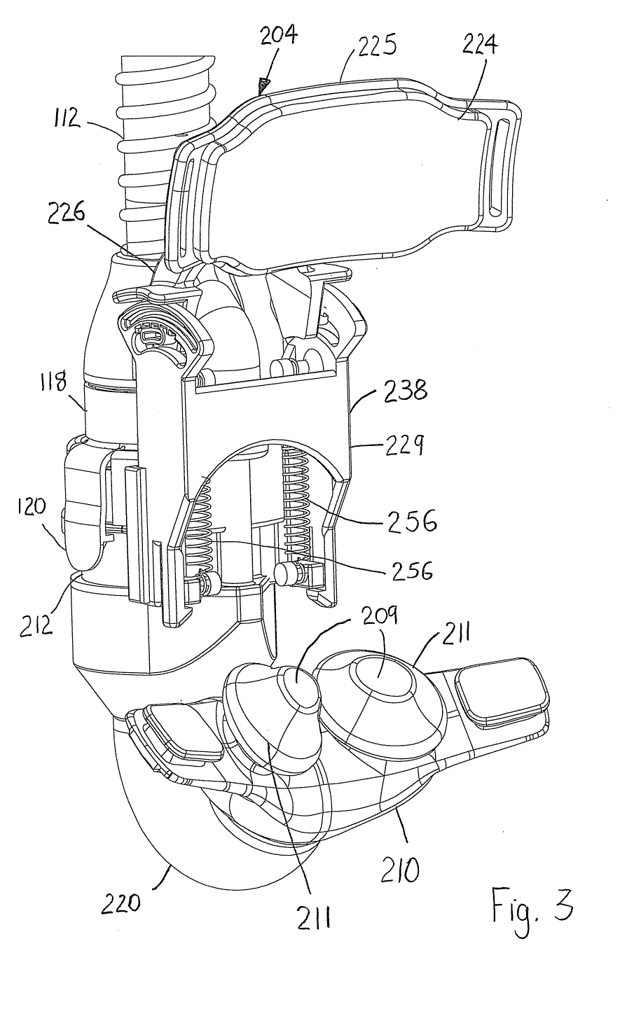

With this brief overview, a nares mask and support apparatus in accordance with various embodiments of the present invention will now be described. FIG. 2 illustrates a partial section view of the nares mask 202 and associated support 204 of the exemplary apparatus 200. As shown in this view, the hose 112 may connect to a housing 206 of the mask 202 as well as to the blower 102 (see FIG. 1). As stated above, when so connected, a lumen 116 defined by the hose 112 may be in fluid communication with the passageway 208 defined by the housing 206. The passageway 208 may also extend through to openings 209 formed in a nares interface 210 of the mask as also shown in FIG. 3. As a result, pressurized air may be delivered from the blower 102 (see FIG. 1A) to the nares 114 of the user during blower operation.

The nares interface 210 may include naris inserts configured to engage, e.g., abut or fit into, the user's nares. Each insert may form a pillow 211 as shown in FIG. 3 made of a deformable material such as silicone or the like. In some embodiments, when the system is pressurized, pressurized air within the passageway 208 of the housing and nares interface causes the pillows 211 to inflate, extend, or deform to the nares shape and provide a sealing surface to abut the areas in and/or around each nare. Once again, the opening 209 formed in each insert and surrounded by the pillow 211 may be in fluid communication with the passageway 208 of the housing 206 such that pressurized air from the blower 102 (see FIG. 1A) may pass into the nares.

With continued reference to FIGS. 1A-3, the hose 112 may include a hose connector 118 that may be attached or integrally formed with the end of the hose. The connector 118 may be configured to selectively and securely engage a receiving mask connector 212, e.g., with the use of snap fit tabs 120 (only one visible in FIGS. 1A and 3). By permitting selective detachment of the hose 112 from the mask 202, the components of the system may be easily cleaned and inspected.

In one embodiment, a heat and moisture exchange (HME) element or cartridge 214 (see FIG. 2) may be located within the passageway 208 and/or the lumen 116 of the hose 112. The HME cartridge 214 may function as an “artificial nose,” whereby it may warm and humidify incoming air by absorbing exhaled moisture during the previous breath. In the illustrated embodiment, the HME cartridge is located in the connector 118 downstream from a vent 122. To facilitate insertion and removal of the HME cartridge 214 from the passageway 208, the cartridge may include a grip 216. When the connectors 118 and 212 are detached, the grip 216 may be positioned for easy grasping within the connector 118. Exemplary HME materials and alternative HME configurations are described in more detail in PCT published application WO 2010/096467.

The blower 102 (e.g., the housing of the blower; see FIG. 1A) may also include a pressure sensor, e.g., to detect pressure at the mask. Accordingly, a sensor tube 218 may extend from the blower 102 to the mask as generally indicated in FIG. 2. In the illustrated embodiment, the tube 218 is located within the lumen 116 of the hose 112. However, in other embodiments, the sensor tube may be located external to the lumen, e.g., alongside the hose or within a hose wall, without departing from the scope of the invention.

The housing 206 of the nares mask 202, which may be positionable adjacent the user's face, defines an interface between the hose 112 and the nares interface 210 as perhaps best shown in FIG. 2. While the housing 206 could be configured as a singular element, it is, in the illustrated embodiment, an assembly of multiple components. For instance, the housing 206 may include both a generally rigid body 207 (defining the mask connector 212 at one end), as well as a flexible or rigid elbow 220 (attached to an end of the body opposite the connector 212). The elbow 220 may, in turn, be securely coupled to the nares interface 210 as shown in FIG. 1A and 2.

Although not illustrated, other embodiments of the housing 206 may include one or more adapters that may be selectively placed between the elbow 220 and the nares interface 210. Such adapters may permit usage of the apparatus 200 with nares interfaces from various manufacturers without necessitating replacement of the elbow 220. Alternatively, other embodiments could forego adapters and instead entirely replace the housing 206 with an alternative housing to accommodate a nares seal of a different design/manufacturer.

To locate and support the mask 202 relative to the user, an anchor, e.g., the support 204, may be provided. The support 204 is, in one embodiment, secured to the scalp, e.g., to the forehead, of the user 104, with one or more elastic bands 222 (shown only in FIGS. 1A and 1B) that may extend circumferentially around the head. For comfort, the support 204 may include a soft or compressible pad 224 to rest against the scalp (e.g., forehead) as shown in FIGS. 2 and 3. The pad 224 may conform in shape to the forehead when the support is correctly attached to the head.

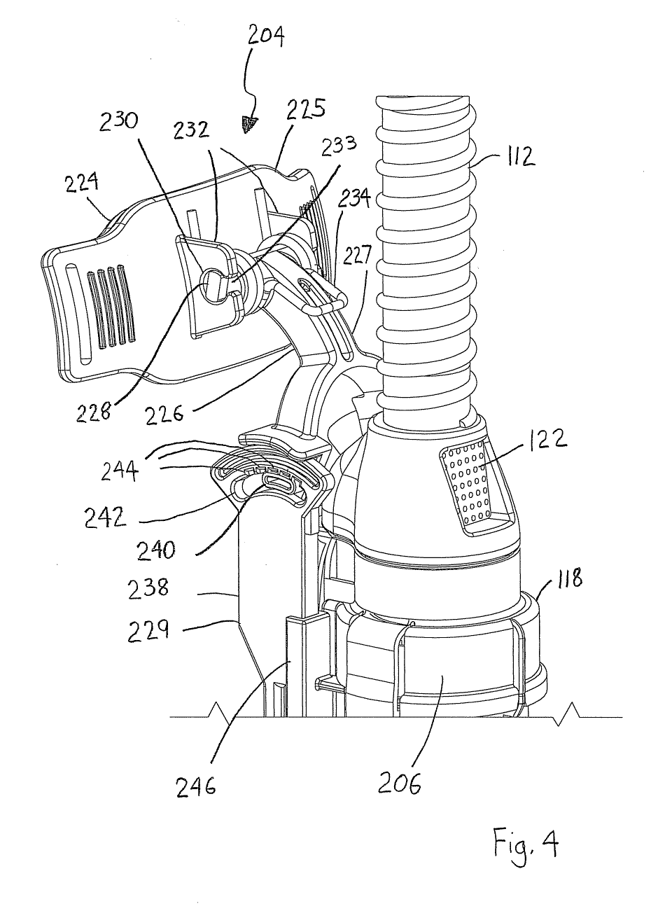

FIG. 4 is an enlarged view of a portion of the support 204. As shown in this view, the support 204 may provide a base 225 that pivotally supports an arm 226 extending from the anchor or base to the housing 206. While the arm 226 may be configured as a single, unitary link, it may, in one embodiment, be configured as a multi-link assembly. For example, the arm 226 may include a first (e.g., upper) end or portion 227 attached to the base, and a second (e.g., lower) end or portion 229 attached to the housing 206.

The first portion 227 of the arm 226 may be generally Y-shaped and form stub shafts 228 (only one visible in FIG. 4) at one end that are received in corresponding apertures 230 formed in tabs 232 protruding from the base 225 of the support 204. In one embodiment, the stub shafts 228 are generally rectangular in cross section such that they may be positioned within their respective apertures 230 by aligning the stub shafts with a slot 233 formed in each tab 232. Once inserted, the arm 226, e.g., stub shafts 228, may be rotated to an operational position whereby the shafts 228 are then retained for pivotal motion only.

A first biasing element, e.g., a torsion spring 234, may be provided to bias the arm 226 in a direction 236 that is towards a face 103 of the user (see FIG. 2). The spring 234 may, in the illustrated embodiment, be interposed or otherwise act between the base 225 and an upper surface (e.g., first portion 227) of the arm 226 as shown in FIG. 4.

The second or lower portion 229 of the arm 226 may operatively connect to the housing 206 of the nares mask 202 as further described below. As best shown in FIG. 4, the lower portion 229 of the arm 226 may form a separate frame 238 that is adjustable (e.g., pivotable) relative to the first or upper portion 227 of the arm. Such adjustability between the two portions of the arm 226 may allow the apparatus 200 to conform to the particular anatomical variations present in a broad user population. Once again, while shown and described as having a two-part arm configuration, other embodiments using a singular component to form the arm are also contemplated.

In the illustrated embodiment, adjustability of the arm 226 is provided by tabs 240 formed on the divergent Y-shaped lower ends of the first portion 227 of the arm. The tabs 240 may translate within a curved window 242 as shown in FIG. 4. Protrusions (not shown) formed on each tab 240 may engage one of a plurality of slots 244 formed in the lower portion 229 (i.e., formed in the frame 238), such that the two components become locked relative to one another. To adjust the frame 238 relative to the first or upper portion 227 of the arm, the user may apply a compressive force to the diverging Y-shaped lower ends of the upper portion 227. This will displace the Y-shaped ends inwardly, withdrawing the tabs 240 and their protrusions (not shown) from the slots 244 and permitting translation of the tabs 240 within the curved windows 242. Upon reaching the desired location, the compressive force may be released, allowing the protrusions to enter a new slot 244, which once again locks the frame 238 relative to the first or upper portion 227 of the arm 226. Thus, the frame may be secured in two or more locations relative to the first portion of the arm.

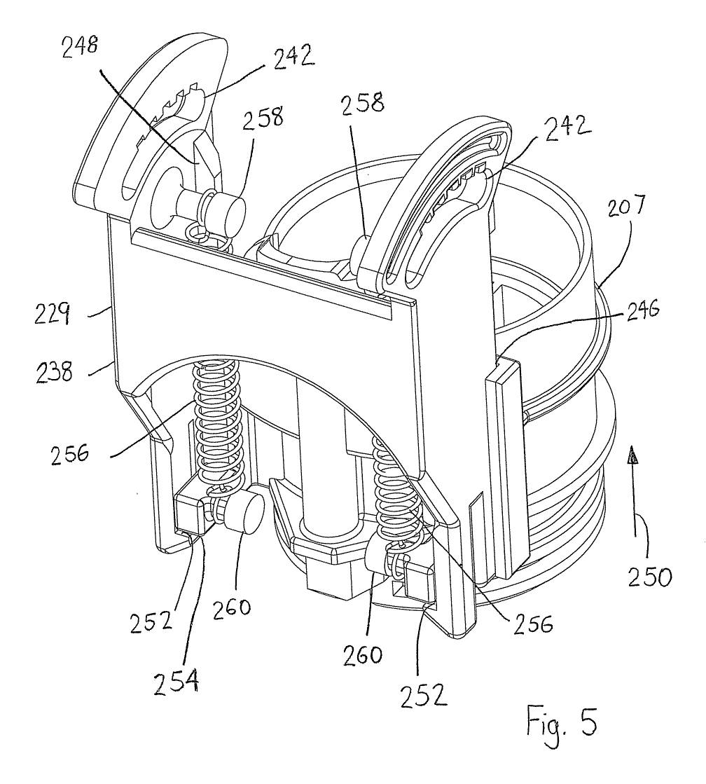

FIG. 5 illustrates an exemplary configuration for attachment of the lower portion 229 of the arm 226 (e.g., the frame 238) to the mask 202 (for clarity, some components of the mask and support apparatus are removed from this view). As shown in the view, the frame 238 may, in one embodiment, attach to the body 207 of the housing 206. To accommodate such attachment, the body 207 may define channels 246 that receive engaging slides 248 formed on the frame 238. Once the channels 246 are engaged with the slides 248, relative motion between the frame 238 and body 207 of the housing is generally limited to only translation in a vertical direction 250 (as viewed in FIGS. 2 and 5). Stops 252 may be provided on the frame 238 to abut stops 254 on the body 207 to limit separation of the body from the frame after the two parts are assembled.

A second biasing element, e.g., extensions springs 256, may be interposed or otherwise attached between pins 258 on the frame 238 of the arm 226 (e.g., on the second portion 229) and corresponding pins 260 on the body 207 of the housing 206. By incorporating the springs 256, a biasing force may be provided that biases the body 207 of the housing (and thus the nares interface 210), relative to the frame 238, in the direction 250, i.e., in a direction generally parallel to the face 103 and towards the user's forehead, e.g., towards the base 225 (see also FIG. 2).

While exemplary embodiments are described and illustrated herein as incorporating specific biasing elements (e.g., a first biasing element configured as a torsion spring and a second biasing element configured as an extension spring), such a configuration is not limiting. For instance, one or both of the first and second biasing elements could be configured as a helical compression element, helical tension element, torsion spring, clock spring, volute spring, wire form element, or similar device capable of providing the desired biasing force. Moreover, the first and second biasing elements could be constructed of most any material including, but not limited to, metal, plastic, composite, elastic fabric (e.g., a bungee cord component, which could provide bias force adjustment via a repositionable hook-and-loop fastener), or the like.

Thus, as shown in FIG. 2, the first biasing element (torsion spring 234) may bias the mask 202 (and thus the nares interface 210 via the arm and the housing), relative to the base, in a direction 236 that is towards the face 103 of the user. While this is a pivoting force, it may effectively act in a localized direction that is generally orthogonal to a plane of the user's face 103. Moreover, one or more second biasing elements (extension springs 256) may bias the mask 202/nares interface 210, relative to the base, in a direction 250 that is generally parallel to the face 103 and towards the user's forehead. As a result, the apparatus (e.g., the biasing elements) may be configured to provide a resultant biasing or retention force 262 (see FIG. 2) having a direction that is generally aligned with the nares, e.g., aligned with a composite axis 264 of the nares 114. In addition to providing potentially increased comfort, such an apparatus may provide adequate mask retention without the need for additional facial-contacting straps or, alternatively, minimize the number of facial-contacting straps needed, e.g., support and biasing of the nares mask may be accomplished without contacting the user's face at locations other than at or near the nares. Even if facial contacting straps are used, masks in accordance with embodiments of the present invention may provide a more consistent force between the seals and nares to assist in reducing or eliminating leaks, and may do so with potentially smaller strap forces.

In embodiments where the CPAP blower is located remotely to the user (see e.g., the blower 1102 in FIG. 1B), the support 204 may provide additional benefits. For instance, a rigid or flexible tether 266 or similar element may be connected to the anchor or support 204 at one end and to the hose 1112 at an opposite end to at least partially constrain the tube and reduce tube-induced forces acting upon the mask. The tether 266 could either be rigid to achieve hose position stability, or could be flexible or semi-rigid to reduce forces from acting on the mask. Stated alternatively, the single anchor 204 may reduce or eliminate the need for a separate head-mounted tube restraint, thereby minimizing or eliminating the need for head straps associated with such a restraint. Various conventional head and face straps could, however, be used in combination with embodiments of the present invention, where such straps are deemed beneficial, without departing from the scope of the invention.

The complete disclosure of the patents, patent documents, and publications cited in the Background, the Detailed Description of Exemplary Embodiments, and elsewhere herein are incorporated by reference in their entirety as if each were individually incorporated.

Illustrative embodiments of this invention are discussed and reference has been made to possible variations within the scope of this invention. These and other variations, combinations, and modifications will be apparent to those skilled in the art without departing from the scope of the invention, and it should be understood that this invention is not limited to the illustrative embodiments set forth herein. Accordingly, the invention is to be limited only by the claims provided below and equivalents thereof.

What is claimed is:

1. A nares mask and support apparatus comprising:
a mask comprising a nares interface configured to engage nares of a user, wherein the mask defines a passageway configured to deliver pressurized air into the nares;
an anchor configured to be secured to a scalp of the user;
an arm extending from the anchor to a housing of the mask;
a first biasing element operatively coupled to the arm, the first biasing element configured to bias the interface in a direction towards a face of the user; and
a second biasing element operatively coupled to the housing, the second biasing element configured to bias the interface in a direction generally parallel to the face and towards a forehead of the user.

2. The apparatus of claim 1, wherein the first biasing element comprises a spring positioned between the anchor and the aim.

3. The apparatus of claim 1, wherein the second biasing element comprises a spring positioned between the arm and the housing.

4. The apparatus of claim 1, further comprising a hose having a first end attachable to the housing, and a second end attachable to a source of pressurized air.

5. The apparatus of claim 4, wherein the hose is partially restrained by a tether attached to the anchor.

6. The apparatus of claim 1, wherein the first and second biasing elements provide a resultant biasing force in a direction aligned with a composite axis of the nares of the user.

7. The apparatus of claim 1, further comprising a heat and moisture exchange element positioned within the passageway.

8. A nares mask and support apparatus comprising:
a mask housing operable for positioning adjacent a user's face, the housing coupled to a nares interface that is configured to engage nares of the user, wherein the housing and the interface together define a passageway configured to deliver pressurized air into the nares;
a base configured to be secured to a forehead of the user;
an arm having a first portion attached to the base and a second portion operatively attached to the housing;
a first spring interposed between the base and the first portion of the arm, the first spring operable to bias the arm and the housing towards the user's face; and
a second spring interposed between the second portion of the arm and the housing, the second spring configured to bias the housing in a direction parallel to the face and towards the base.

9. The apparatus of claim 8, wherein the second portion of the arm comprises a frame pivotally attached to the first portion of the arm.

10. The apparatus of claim 9, wherein the housing translates relative to the frame.

11. The apparatus of claim 9, wherein the frame is configured to be secured in two or more locations relative to the first portion of the arm.

12. The apparatus of claim 8, further comprising a tether extending between the base and a hose connected to the mask housing.

13. The apparatus of claim 8, further comprising a compressible pad attached to the base, the pad configured to conform in shape to the forehead of the user.

14. The apparatus of claim 8, further comprising one or more elastic bands configured to extend circumferentially around a head of the user and attach to the base.

15. The apparatus of claim 8, wherein the first spring comprises a torsion spring.

16. The apparatus of claim 8, wherein the second spring comprises an extension spring.

17. A method for supporting a nares mask relative to a head of a user, the method comprising:
placing a base of an anchor against a forehead of the user, the anchor further comprising an arm pivotally attached to the base and extending away therefrom, the arm coupled at an opposite end to a housing of the nares mask;
inserting a nares interface into nares of the user;
biasing the nares interface in a direction generally orthogonal to and towards a face of the user; and
biasing the nares interface in a direction generally parallel to the user's face and towards the forehead.

18. The method of claim 17, further comprising securing the anchor to the forehead with one or more bands configured to extend circumferentially around the head and attach to the anchor.

19. The method of claim 17, wherein biasing of the nares interface comprises biasing the nares interface without contacting the user's face at a location other than at or near the nares.
